(19) United States
(12) Patent Application Publication
(10) Pub. No.: US 2007/0260132 A1
(43) Pub. Date: Nov. 8, 2007

(54) Method and apparatus for processing signals reflecting physiological characteristics from multiple sensors

(76) Inventor: Bernhard B. Sterling, Danville, CA (US)

Correspondence Address: Ralph C. Francis;Francis Law Group, 1942 Embarcadero, Oakland, CA, 94606, US

(21) Appl. No.: 11/418,937

(22) Filed: May 4, 2006

(51) Int. Cl. A61B 5/00

(52) U.S. Cl. 600/336; 600/323

(57) ABSTRACT

The invention comprises a method and apparatus for processing signals reflecting a physiological characteristic by performing an error minimizing mathematical combination between signals from at least two independent sensors. For example, the intensity of light is detected following tissue absorption at two wavelengths and the signals are corrected. Preferably, corrected intensity signals are derived by orthogonal regression. In one embodiment, the method and apparatus are used to determine arterial oxygen saturation.

### FIELD OF THE PRESENT INVENTION

The present invention relates to the field of signal processing. More specifically, the invention relates to a method for processing signals reflecting physiological characteristics.

### BACKGROUND OF THE INVENTION

Physiological monitoring systems and apparatus, which are adapted to acquire signals reflecting physiological characteristics, are well known in the art. The physiological characteristics include, for example, heart rate, blood pressure, blood gas saturation (e.g., oxygen saturation) and respiration rate.

The signals acquired by the noted physiological monitoring systems and apparatus are however composite signals, comprising a desired signal portion that directly reflects the physiological process that is being monitored and an undesirable signal portion, typically referred to as interference or noise. The undesirable signal portions often originate from both AC and DC sources. The DC component, which is easily removed, results from the transmission of energy through differing media that are of relatively constant thickness within the body (e.g., bone, tissue, skin, blood, etc.).

Undesirable AC components of the acquired signal correspond to variable or erratic noise and interference, and thus have been conventionally quite difficult to characterize and remove.

One example of a physiological monitoring apparatus, wherein the measured signal can, and in many instances will, include undesirable signal components, is a pulse oximeter.

Pulse oximeters typically measure and display various blood constituents and blood flow characteristics including, but not limited to, blood oxygen saturation of hemoglobin in arterial blood, the volume of individual blood pulsations supplying the flesh and the rate of blood pulsations corresponding to each heartbeat of the patient. Illustrative are the apparatus described in U.S. Pat. Nos. 5,193,543; 5,448,991; 4,407,290; and 3,704,706.

As is well known in the art, a pulse oximeter passes light through human or animal body tissue where blood perfuses the tissue, such as a finger, an ear, the nasal septum or the scalp, and photoelectrically senses the absorption of light in the tissue. The amount of light absorbed is then used to calculate the amount of blood constituent being measured.

Two lights having discrete frequencies in the range of about 650-670 nm in the red range and about 800-1000 nm in the infrared range are typically passed through the tissue. The light is absorbed by the blood in an amount representative of the amount of the blood constituent present in the blood. The amount of transmitted light passed through the tissue varies in accordance with the changing amount of blood constituent in the tissue and the related light absorption.

The output signal from the pulse oximeter, which is sensitive to the arterial blood flow, contains a component that is a waveform representative of the patient's blood gas saturation. This component is referred to as a “plethysmographic wave or waveform” (see curve P in FIG. 1).

The plethysmograph signal (and the optically derived pulse rate) may however be subject to irregular variants that interfere with the detection of the blood constituents. The noise, interference and other artifacts can, and in many instances will, cause spurious pulses that are similar to pulses caused by arterial blood flow. These spurious pulses, in turn, may cause the oximeter to process the artifact waveform and provide erroneous data.

Several signal processing methods (and apparatus) have been employed to reduce the effects of undesirable signal components on the measured signal and, hence, the derived plethysmograph waveform. Illustrative are the methods and apparatus disclosed in U.S. Pat. No. 4,934,372, which correlate a subject's electrocardiogram waveform with the acquired signal to identify desired portions of the signal to more accurately detect blood constituents.

Similarly, U.S. Pat. Nos. 5,490,505, 6,036,642, 6,206,830, and 6,263,222, all disclose signal processors that generate either a noise reference or a signal reference that is used to drive a correlation canceler and generate a waveform that approximates either the desired or undesired component of the acquired signal. A primary intended application of the noted signal processors is the measurement of blood oxygen saturation in a manner that minimizes the effect of motion artifacts. However, a consequence of the process used to generate the reference is that a third optical signal must be acquired to provide ratiometric calculation of saturation.

Accordingly, each of the noted prior art references require the use of a reference signal to help measure blood oxygen saturation. As such, these systems are unable to process signals using information from a single pulse wave. Further, the noted references are primarily concerned with filtering out motion artifacts. Therefore, these references are not tailored to the removal of undesired signal components that arise from other sources.

It is therefore an object of the present invention to provide a cost effective, reliable means of determining a physiological characteristic by detecting a minimum number of signals from two or more independent sensors.

A further object of the invention is to provide a method for processing signals reflecting a physiological characteristic by applying an orthogonal regression technique to improve the signal to noise ratio.

It is another object of the invention to provide a method for processing signals reflecting a physiological characteristic that does not require correlation canceling.

Another object of the invention is to provide a method for processing signals reflecting a physiological characteristic that minimizes undesirable signal components.

It is yet another object of the invention to provide a method and apparatus for correcting signals reflecting a physiological characteristic that does not require a pulse waveform model or the use of data from preceding pulse waveforms.

Yet another object of the invention is to provide a method and apparatus for correcting signals reflecting a physiological characteristic using data from a single pulse.

Another object of the invention is to combine signals from two or more independent oximetry sensors to provide a signal having enriched oximetry content.

A further object of the invention is to provide a method and apparatus for determining arterial oxygen saturation with improved accuracy.

Another object of the invention is to provide a method for improving an oximetry signal derived from two or more sensors using weighted averaging by regression to maximize the oximetry signal.

Yet another object of the invention is to provide a method and apparatus for correcting signals reflecting a physiological characteristic including cardiac output, blood pressure, ECG, blood pH, hemoglobin concentration or glucose concentration.

### SUMMARY OF THE INVENTION

In accordance with the above objects and those that will be mentioned and will become apparent below, the invention includes a device for the monitoring of a physiological characteristic of a patient's blood, comprising i) a first sensor having a first radiation emitter that emits light at a first wavelength, a second radiation emitter that emits light at a second wavelength and a radiation detector configured to receive light at the first and second wavelengths after absorbance through the patient's blood and provide a first received intensity signal and a second received intensity signal corresponding to the first and second received wavelengths, ii) a second sensor having a first radiation emitter that emits light at a first wavelength, a second radiation emitter that emits light at a second wavelength and a radiation detector configured to receive light at the first and second wavelengths after absorbance through the patient's blood and provide a first received intensity signal and a second received intensity signal corresponding to the first and second received wavelengths, and iii) a controller for computing the physiological characteristic of the patient's blood from a first corrected intensity signal from the first and second sensors and a second corrected intensity signal from the first and second sensors; wherein the corrected intensity signals are derived from performing an error minimizing mathematical combination between a) the first received intensity signal from the first sensor and the first received intensity signal from the second sensor, b) the second received intensity signal from the first sensor and the second received intensity signal from the second sensor, c) the first received intensity signal from the first sensor and the second received intensity signal from the first sensor, and d) the first received intensity signal from the second sensor and the second received intensity signal from the second sensor.

In one embodiment of the invention, the error minimizing mathematical combination is orthogonal regression.

In one embodiment of the invention, the first and second corrected intensity signals are derived from a weighted average of the first and second received intensity signals.

In one embodiment of the invention, the physiological characteristic is arterial oxygen saturation.

Preferably, the first wavelength is in the range of approximately 650-670 nm n. Also preferably, the second wavelength is in the range of 800-1000 nm.

In one aspect of the invention, a ratio of logarithms of the first and second corrected intensity signals is related to reference oxygen saturation to determine the physiological characteristic.

Preferably, the first and second corrected intensity signals have an improved signal to noise ratio.

Also preferably, a difference between the received intensity signals and the corrected intensity signals substantially corresponds to undesirable signal components.

In one embodiment of the invention, the controller indexes the received intensity signals to the patient's pulse amplitude.

In another embodiment, the controller indexes the received intensity signals to the patient's pulse amplitude and the controller averages the ratio of logarithms from the first sensor is averaged with the ratio of logarithms from the second sensor.

In a further aspect of the invention, the ratio of logarithms from the first sensor is averaged with the ratio of logarithms from the second sensor when a difference between the ratio of logarithms is below a desired acceptance criterion.

The invention also comprises a method for processing signals reflecting a physiological characteristic of a patient's blood, including the steps of i) coupling a first and second oximeter sensor arrangement to independent tissue regions of the patient, ii) passing first and second lights through the patient's tissue region at each sensor arrangement, wherein the first light is substantially in a red light range and the second light is substantially in an infrared light range, iii) detecting the first and second lights absorbed by the tissue region and providing a first received intensity signal and a second received intensity signal corresponding to the absorbed first and second lights with each sensor arrangement, and iv) computing the physiological characteristic of the patient's blood from first corrected intensity signals from each sensor arrangement and second corrected intensity signals from each sensor arrangement determined by performing an error minimizing mathematical combination between a) the first received intensity signal from the first sensor arrangement and the first received intensity signal from the second sensor arrangement, b) the second received intensity signal from the first sensor arrangement and the second received intensity signal from the second sensor arrangement, c) the first received intensity signal from the first sensor arrangement and the second received intensity signal from the first sensor arrangement, and d) the first received intensity signal from the second sensor arrangement and the second received intensity signal from the second sensor arrangement.

In one embodiment of the invention, the error minimizing mathematical combination is orthogonal regression.

In one embodiment of the invention, the corrected intensity signals are derived from a weighted average of the received intensity signals.

Preferably, the physiological characteristic is arterial oxygen saturation.

Also preferably, a ratio of logarithms of the corrected intensity signals is related to reference oxygen saturation to determine the physiological characteristic.

In one embodiment of the invention, the method also includes the step of indexing the received intensity signals to the patient's pulse amplitude.

In another embodiment, the invention includes the steps of indexing the received intensity signals to the patient's pulse amplitude and averaging the ratio of logarithms from the first sensor is averaged with the ratio of logarithms from the second sensor.

In a further aspect of the invention, the ratio of logarithms from the first sensor is averaged with the ratio of logarithms from the second sensor when a difference between the ratio of logarithms is below a desired acceptance criterion.

In one embodiment of the invention, the device also includes at least one additional sensor having a first radiation emitter that emits light at a first wavelength, a second radiation emitter that emits light at a second wavelength and a radiation detector configured to receive light at the first and second wavelengths after absorbance through the patient's blood and provide a first received intensity signal and a second received intensity signal corresponding to the first and second received wavelengths and wherein the controller computes the physiological characteristic of the patient's blood corrected intensity signals including corrected intensity signals from the additional sensor.

The invention also comprises a method for processing signals reflecting a physiological characteristic of a patient's blood, including the steps of i) coupling a first and second oximeter sensor arrangement to independent tissue regions of the patient, ii) passing first and second lights through the patient's tissue region at each sensor arrangement, wherein the first light is substantially in a red light range and the second light is substantially in an infrared light range, iii) detecting the first and second lights absorbed by the tissue region and providing a first received intensity signal and a second received intensity signal corresponding to the absorbed first and second lights with each sensor arrangement, and iv) computing the physiological characteristic of the patient's blood from first corrected intensity signals from each sensor arrangement and second corrected intensity signals from each sensor arrangement determined by performing orthogonal regression between a) the first received intensity signal from the first sensor arrangement and the first received intensity signal from the second sensor arrangement, b) the second received intensity signal from the first sensor arrangement and the second received intensity signal from the second sensor arrangement, c) the first received intensity signal from the first sensor arrangement and the second received intensity signal from the first sensor arrangement, and d) the first received intensity signal from the second sensor arrangement and the second received intensity signal from the second sensor arrangement.

The invention also comprises a method for processing signals reflecting a physiological characteristic of a patient's blood, including the steps of i) coupling a first and second physiological sensor arrangement to independent tissue regions of the patient, ii) detecting a signal reflecting the physiological characteristic with each sensor arrangement, and computing the physiological characteristic of the patient's blood from corrected signals from each sensor arrangement determined by performing an error minimizing mathematical combination between the signal from the first sensor arrangement and the signal from the second sensor arrangement.

### BRIEF DESCRIPTION OF THE DRAWINGS

Further features and advantages will become apparent from the following and more particular description of the preferred embodiments of the invention, as illustrated in the accompanying drawings, and in which like referenced characters generally refer to the same parts or elements throughout the views, and in which:

FIG. 1 is a graphical illustration of an r-wave portion of an electrocardiogram waveform and the related plethysmographic waveform;

FIG. 2 is a schematic illustration of a pulse oximeter apparatus, according to the invention;

FIGS. 3 and 4 are graphical illustrations of red and infrared optical signals taken from independent sensors, according to the invention;

FIGS. 5 and 6 are graphical illustrations of experimental data comparing the red and infrared signals, respectively, from independent sensors A and B to illustrate common cardiac cycle information, according to the invention;

FIGS. 7 and 8 are graphical illustrations of the relationship between the red and infrared signal, respectively, after correction by orthogonal regression between sensors A and B, according to the invention;

FIGS. 9 and 10 are graphical illustrations of the relationship between the red signal from sensor A and the infrared signals from sensors A and B, respectively, after correction by orthogonal regression, according to the invention;

FIGS. 11 and 12 are graphical illustrations of the relationship between the red signal from sensor B and the infrared signals from sensors A and B, respectively, after correction by orthogonal regression, according to the invention;

FIGS. 13 and 14 are graphical illustrations of the red signal of a single pulse from each sensor before and after correction by orthogonal regression, respectively, according to the invention;

FIGS. 15 and 16 are graphical illustrations of the ratio of logarithms of the acquired data from each sensor before and after correction by orthogonal regression, respectively, according to the invention;

FIGS. 17 and 18 are graphical illustrations of the ratio of logarithms compared to pulse amplitude from each sensor before and after correction by orthogonal regression, respectively, according to the invention;

FIG. 19 is a graphical illustration of oximetry data from independent sensors showing differences that imply erroneous reference CO-oximeter data acquisition, according to the invention; and

FIG. 20 is a graphical illustration of oximetry data from independent sensors showing differences that imply a physiological condition, according to the invention.

### DETAILED DESCRIPTION OF THE INVENTION

Before describing the present invention in detail, it is to be understood that this invention is not limited to particularly exemplified materials, methods or structures as such may, of course, vary. Thus, although a number of materials and methods similar or equivalent to those described herein can be used in the practice of the present invention, the preferred materials and methods are described herein.

It is also to be understood that the terminology used herein is for the purpose of describing particular embodiments of the invention only and is not intended to be limiting.

Unless defined otherwise, all technical and scientific terms used herein have the same meaning as commonly understood by one having ordinary skill in the art to which the invention pertains.

Further, all publications, patents and patent applications cited herein, whether supra or infra, are hereby incorporated by reference in their entirety.

Finally, as used in this specification and the appended claims, the singular forms “a, “an” and “the” include plural referents unless the content clearly dictates otherwise.

### Definitions

The term “signal”, as used herein, is meant to mean and include an analog electrical waveform or a digital representation thereof, which is collected from a biological or physiological sensor.

The term “desired signal component”, as used herein, is meant to mean and include the portion of a signal that directly corresponds to the biological or physiological function being monitored.

The term “motion artifact”, as used herein, is meant to mean and include variability in a signal due to changes in the tissue being monitored that are caused by muscle movement proximate to the oximeter sensor.

The term “undesirable signal component”, as used herein, is meant to mean and include any portion of a signal that does not correspond to the biological or physiological function being monitored. As such, the term includes, without limitation, noise, interference, and other variables that hinder the measurement of the biological or physiological function. Generally, motion artifacts are not the subject of this invention.

The terms “patient” and “subject”, as used herein, is meant to mean and include humans and animals.

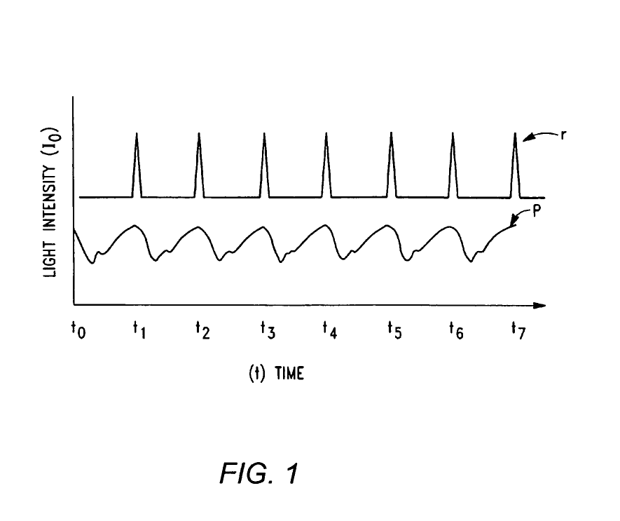

Referring first to FIG. 1, there is shown a graphical illustration of an “r-wave” portion of an electrocardiogram (ECG) waveform (designated “r”) and the related plethysmographic waveform (designated “p”). As will be appreciated by one having ordinary skill in the art, the ECG waveform comprises a complex waveform having several components that correspond to electrical heart activity. The QRS component relates to ventricular heart contraction.

The r-wave portion of the QRS component is typically the steepest wave therein, having the largest amplitude and slope, and can be used for indicating the onset of cardiovascular activity. The arterial blood pulse flows mechanically and its appearance in any part of the body typically follows the R wave of the electrical heart activity by a determinable period of time that remains essentially constant for a given patient. See, e.g., Goodlin et al., Systolic Time Intervals in the Fetus and Neonate, Obstetrics and Gynecology, Vol. 39, No. 2, (February 1972) and U.S. Pat. No. 3,734,086.

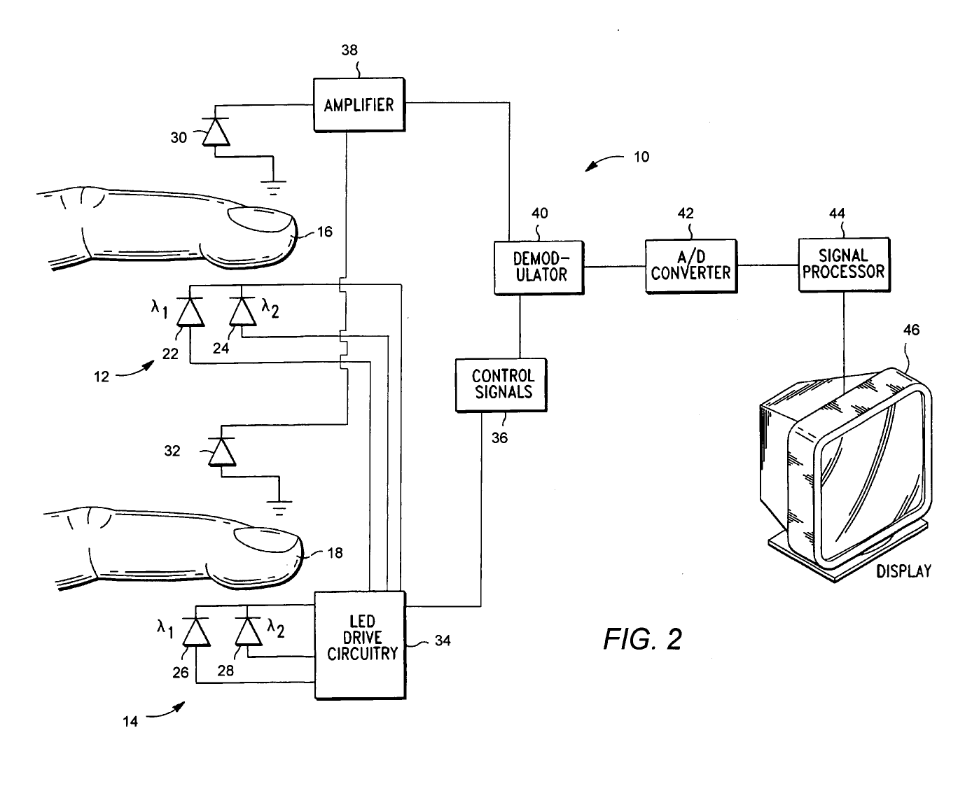

Referring now to FIG. 2, there is shown a schematic illustration of one embodiment of a pulse oximeter apparatus 10 comprising two sensors 12 and 14 that can be employed within the scope of the invention. As discussed above, conventional pulse oximetry methods and apparatus typically employ a sensor using two lights; a first light having a discrete wavelength in the range of approximately 650-670 nm in the red range and a second light having a discrete wavelength in the range of approximately 800-1000 nm. For example, a suitable red LED emits light at approximately 660 nm and a suitable infrared LED emits light at approximately 880 nm.

Sensors 12 and 14 are independent, and can be positioned on fingers 16 and 18 of each hand of a subject, for example. The lights are typically directed through fingers 16 and 18 via emitters 22, 24, 26, and 28 and detected by photo detectors 30 and 32, such as square photodiodes, each with an area of 49 mm². Emitters 22, 24, 26 and 28 are driven by drive circuitry 34, which is in turn governed by control signal circuitry 36. Detectors 30 and 32 are in communication with amplifier 38. In one embodiment, the LEDs are activated at a rate of 8,000 times per second (8 kHz) per cycle, with a cycle comprising red on, quiescent, IR on, quiescent. In the noted embodiment, the total cycle time is 125 microseconds and the LEDs are active for approximately 41.25 microseconds at a time.

The photo detectors 30 and 32 provide output signals that are transmitted to amplifier 38. The signal from amplifier 38 is then transmitted to demodulator 40, which is also synched to control signal circuitry 36. As will be appreciated by one having skill in the art, the output signal from the demodulator 40 is a time multiplexed signal comprising (i) a background signal, (ii) the red light range signal and (iii) the infrared light range signal from each sensor 12 and 14.

The demodulator 40, which is employed in most pulse oximeter systems, removes any common mode signals present and splits the time multiplexed signal into four channels, representing the red voltage (or optical) signal and the infrared voltage (or optical) signal from each sensor.

As illustrated in FIG. 2, the signal from the demodulator 40 is transmitted to analog-digital converter (ADC) 42. The desired computations are performed on the output from the converter 42 by signal processor (DSP) 44 and the results transmitted to display 46. In one embodiment, ADC 42 converts the analog signals into 16-bit signed digital signals at a rate of 8 kHz. Further, DSP 44 preferably notch filters the data at 40 Hz to eliminate power line frequency noise limit high frequency noise from other sources. Also preferably, the DSP then parses each data stream by a factor of 4 to give four digital data streams at a rate of 2 kHz, corresponding to the red and infrared signals from each sensor.

Further details of conventional pulse oximeter components, and related functions, are set forth in U.S. Pat. No. 4,934,372, which is incorporated by reference herein.

As one having ordinary skill in the art will recognize, the teachings of this invention can be extended to include additional sensors if desired. For example, U.S. Pat. Nos. 6,480,729, 6,537,225, 6,594,511, 6,719,705, 6,819,950, and 6,921,367 and U.S. patent application Ser. No. 10/912,721, filed 4 Aug. 2004, all of which are incorporated in their entirety by reference, each disclose methods and apparatus employing multiple sensors that may be practiced with the present invention.

In one embodiment, the system electronics are configured such that emitters 22, 24, 26 and 28 are driven with a variable gain to produce an AC signal (corresponding to the photoplethysmograph pulse waveform) riding on a larger DC signal. The current supplied to the emitters is feedback driven to produce a constant DC signal of approximately 1.25 V, for both the red and infrared signals. The actual DC value is reported continuously. The magnitude of the AC signals is computed relative to the DC signal. The AC component is the signal that is given to the ADC 42 and converted to digital, with the DC signal treated as the “zero point”. This creates a factor of the voltage range of the ADC 42 divided by the dynamic (digital) range of the DSP 44. As one having skill in the art will recognize, actual AC voltage level is computed by multiplying the digital AC counts are multiplied by the voltage conversion factor times the DC voltage.

In conventional pulse oximetry, a single sensor is typically used on one of the two index fingers. Such an oximetry sensor delivers oxygen saturation data having an accuracy of one to two percent. In many situations, the results obtained from a single sensor are adequate for ascertaining the basic oxygenation status of a well-controlled patient. Accordingly, conventional practice does not suggest the use of a second sensor for additional data acquisition given the relatively small gains expected from additional signal averaging given the dominant noise of the equipment and the adequate precision of the sensor.

However, when high performance monitoring equipment and methods are employed that deliver oxygenation status information at significantly higher resolution, data from an additional sensor can provide important information, both from a technical and physiological perspective. Specifically, unexpected diagnostic information can be gained from the simultaneous use of a two or more sensor system through the practice of this invention. There is also a concurrent improvement in signal to noise precision from combining twice as much data. Thus, the invention represents a means of providing patient monitoring with enhanced reliability and a means of gaining additional diagnostic physiological information unavailable with the use of a single sensor system in addition to the use of multiple sensors to improve data precision.

Therefore, according to the invention, a comparison of oximetry data from two independent sites helps determine the undesirable signal components while improving the accuracy of the underlying oxygen saturation measurement. Typically, the technical limitations of the instrumentation can be confirmed to be relatively insignificant. If so, differences between the two sets of data derived from each sensor allow the assessment of additional physiologic parameters. In this manner, the present invention substantially reduces or eliminates the disadvantages and drawbacks associated with convention signal processing systems, apparatus and techniques.

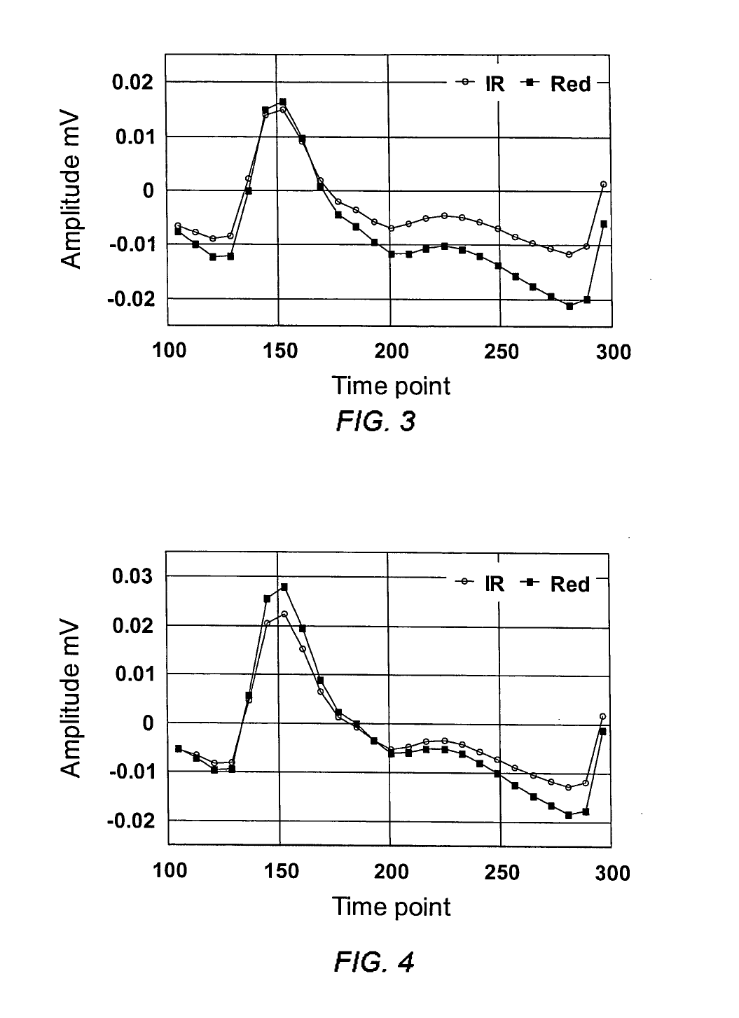

The specifics of this process are discussed below with respect to exemplary signal data obtained from pulse oximeter 10 using two independent sensors A and B corresponding to sensors 12 and 14. In one embodiment, each sensor is attached to the right and left index fingers of a subject. FIGS. 3 and 4 show exemplary data collected during a single pulse, from sensors A and B, respectively. In one embodiment, maximal and minimal amplitudes of the data streams are determined using a comparator on a continuous moving average of 50 samples. Depending upon the application, different sample rates can be used.

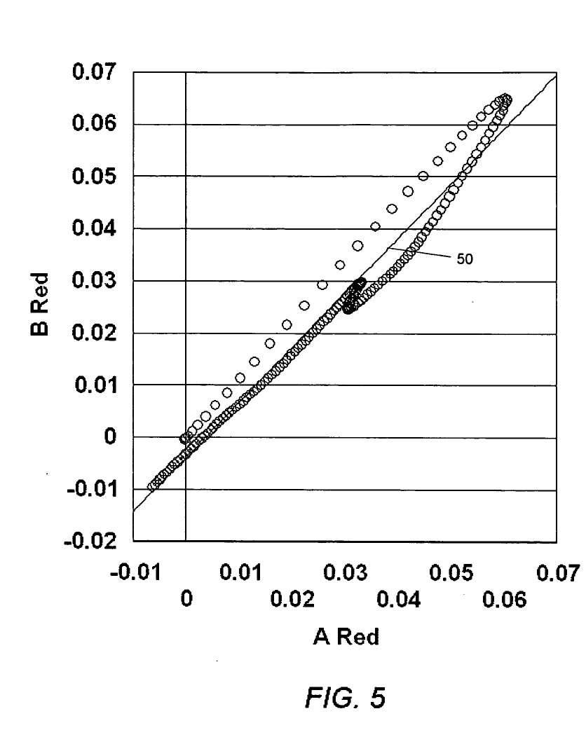

As discussed above, the experimental data collected from independent sensors A and B contains common cardiac cycle information. FIG. 5 shows data corresponding to the red signal from sensor A during a single pulse graphed against the red signal of the independent second sensor B during the same single pulse. As can be seen, the red signal from sensors A and B exhibit a relatively linear relationship, approximated by line 50. This relationship demonstrates the common cardiac cycle information. However, the deviations of the data points from line 50 represent errors in the data that are subject to correction. To obtain the correct offset in the output equation, it is preferable to offset the individual data sets. For example, a preferred offset is achieved by subtracting the required mV to bring both plethysmographic data sets to zero at an identifiable data minimum, such as the ‘trough’ before the coming pulse wave.

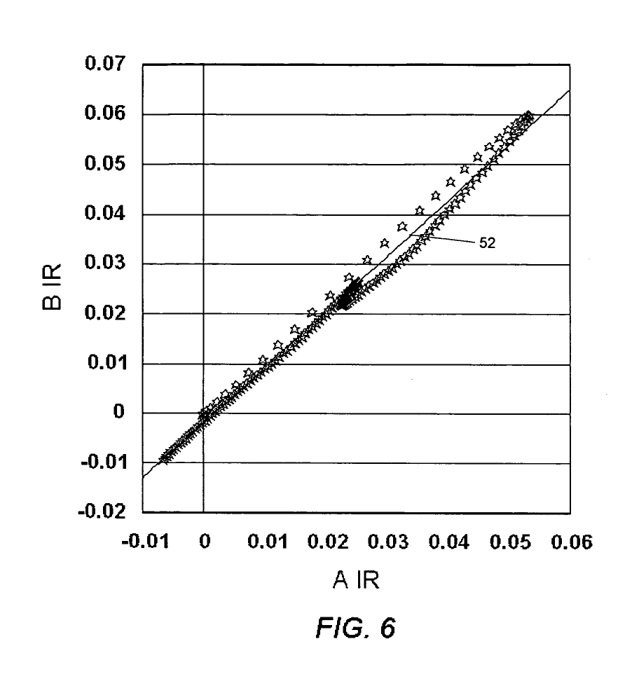

Similarly, FIG. 6 shows data corresponding to the infrared signal from sensor A during a single pulse graphed against data corresponding to the infrared signal of the independent second sensor B during the same single pulse. As with the data from the red signal, the infrared data from sensors A and B exhibit a relatively linear relationship, approximated by line 52, indicating common cardiac cycle information.

Typically, sensor data contains both desirable signal components that reflect physiological characteristics and undesirable noise. Accordingly, maximizing the signal to noise ratio improves the precision of the measurement. There are a number of conventional means for optimizing the signal to noise ratio for two independent data sets. For example, the all data collected at the same time point is simply averaged. Further refinements include applying adjustable weighting factors to favor the better data set before combining or calculating a relationship of the combined data, for example, by a linear least squares fitting routine.

However, for the purpose of optimizing signal to noise, statistically derived parameters of regression and correlation are not important and are not required. Specifically, the goodness-of-the-fit is not an important characteristic and statistical expressions, such as correlation coefficient are not needed because there is no independent arbitrating ‘true’ data set against which the individual data sets can be compared.

Thus, according to the invention as described herein, data sets from two or more independent sensors are consecutively processed using orthogonal regression to maximize signal to noise ratio and improve measurement precision.

In one embodiment, a physiological characteristic of a patient's blood is monitored with two or more independent sensors, each having first and second radiation emitters that emit light at first and second wavelengths, a radiation detector configured to receive light at the first and second wavelengths after absorbance through the patient's blood and provide first and second intensity signals corresponding to the first and second received wavelengths. A controller computes the physiological characteristic of the patient's blood from a corrected first and second intensity signal from each sensor. The intensity signals are corrected by performing an orthogonal regression on the combination of the first signal of the first sensor with the first signal of the second sensor and the second signal of the first sensor with the second signal of the second sensor. A subsequent orthogonal regression is performed on a combination of the first and second signals of the first sensor and a combination of the first and second signals of the second sensor.

As discussed above with reference to FIGS. 5 and 6, the red and infrared signals from independent sensors exhibit a relatively linear relationship. This general relationship for each sensor can be expressed as:
y=mx+b (1)
Specifically, FIG. 5 has an R-square equal to 0.9716 with 175 data points and line 50 corresponds to the equation y=−0.00389+1.05× and FIG. 6 has an R-square equal to 0.992 with 175 data points and line 52 corresponds to the equation y=−0.00199+1.12x.

If the experimental data were perfectly obtained, the slope m would be one and the offset b zero. In practice, one or more sources of noise are contained in the data signal, resulting in the deviations from lines 50 and 52 shown in FIGS. 5 and 6, respectively. The observation of this experimental difference offers several approaches for improving the data, such as correcting the data to the ideal case of y=x, correcting to a semi-empirical case of data fitting while x=0 at y=0 is maintained, or calculating a standard linear regression with floating m and b.

Conventional signal processing employing linear regression minimizes the sum of squares for a data set {(x₁,y₁), . . . , (xₙ, yₙ)}. Geometrically, this calculation corresponds to minimizing the sum of squared lengths of vertical line segments connecting the data points to a single line. Although this method has certain applications, it suffers from the assumption that all errors in the data set are due to errors in Y.

In this invention, orthogonal regression is preferably employed to accommodate situations in which both variables contain errors. Orthogonal regression minimizes the sum of the squared lengths of the shortest lines connecting the data points to a single line. This procedure assumes that the standard error for the X variate is equal to the standard error for the Y variate. If these are not equal, the variates are preferably rescaled to equalize standard errors. After performing the orthogonal regression, the results are preferably scaled back to the original values. In ordinary linear regression, the goal is to minimize the sum of the squared vertical distances between the y data values and the corresponding y values on the fitted line. In contrast, the goal of orthogonal regression is to minimize the orthogonal (perpendicular) distances from the data points to the fitted line.

Orthogonal regression is preferably performed on the red and infrared signals from the independent sensors in the following manner. Although the process is not limited to linear relationships, in one embodiment a regression line L is expressed as:
y=mx+b (2)
with the constants m and b corresponding to slope and offset, respectively. Given the data point x=c, y=d, the equation of the shortest line connecting (c,d) to the line L represented by equation (2) is found from the point-slope form of the line, given that it passes through the data point (c,d), and that it has a slope equal to the negative of the reciprocal of the slope of L. Thus, since the slope of the line L is m, the slope of the desired line L^{⊥}, is −1/m. Accordingly, the line L^{⊥} can be expressed by the equation:
y=−x/m+b (3)

At the data pair (c,d) this relationship becomes:
d=−c/m+b (4)
or
b=d+c/m (5)

Accordingly, the desired line L^{⊥} can therefore be expressed by the equation:
y=−x/m+d+c/m (6)

In order to correct the data pair (c,d) to its corresponding error-free data pair on the line (L), both lines must have equal y and x values where the two lines cross. By setting the y values equal, the following expressions are derived:
−x/m+d+c/m=mx+b (7)
or the combination of:
x=(−b+d+c/m)/(1/m+m) (8)
and
y=(b/m+dm+c)/(1/m+rm). (9)

Preferably, every data pair such as (c,d), is corrected to corresponding values on the regression line y=mx+b by substituting x and y with the known values in (b,c,d, and m) with the equations (8) and (9) above. As desired, this step may include setting slope and offsets to m=1 and b=0 or any other pre-set condition which forces the regression fit through a specific value or maintains a desired slope.

Orthogonal regression in general is a known routine for converting regression algorithms into corresponding orthogonal regression. A general description of its application is given in ACM Transactions on Mathematical Software (TOMS), Vol. 14, pp. 76-87, Issue 1 (March 1988), which is hereby incorporated by reference in its entirety. Further discussion of orthogonal regression can be found in Brown, M. Robust Line Estimation With Errors In Both Variables, J. Am. Star. Assoc., Vol. 377, pp. 71-79 (1982) and Cheng, C. L., and Van Ness, J. Robust Errors-In-Variables Regression, Tech. Rep., Mathematical Sciences Program, Univ. of Texas at Dallas, Richardson, Tex. (1987), both of which are incorporated herein in their entirety by reference.

Thus, orthogonal regression is a robust error minimization technique that is based on a defined mathematical relationship, such as a linear function, and is especially useful for minimizing errors in a relationship of two variables where each variable contains its own significant error or noise. In contrast, standard regression is best suited to situations wherein one of the two variables is substantially noise-free and can thus be considered a reference.

### EXAMPLE

The following example is given to enable those skilled in the art to more clearly understand and practice the present invention. It should not be considered as limiting the scope of the invention, but merely as being illustrated as representative thereof.

To illustrate an embodiment of the present invention, oximetry data was collected and corrected with respect to reference oxygen saturation. The data was collected from 8 adult volunteers. For the study, a catheter was placed into a radial artery of each subject. A Nellcor N-200 pulse oximeter was used as a reference device, and also for clinically monitoring the subject. Each subject was given varying inspired concentrations of oxygen in order to produce arterial hemoglobin oxygen saturations in the approximate range of 70-100%. Blood samples were drawn from the arterial catheter simultaneously with readings of oxygen saturation, and immediately analyzed. Data were collected of both the waveform being analyzed, as well as computed intermediate steps. The arterial blood sample was analyzed on two separate blood-gas analyzers by Radiometer. The functional saturation of hemoglobin was computed as oxyhemoglobin/(total hemoglobin). That is, all non-oxyhemoglobin species were included in total hemoglobin. At all saturations and for all human study subjects, the reference values for the algorithmically computed values were the average readings from two CO-oximeters.

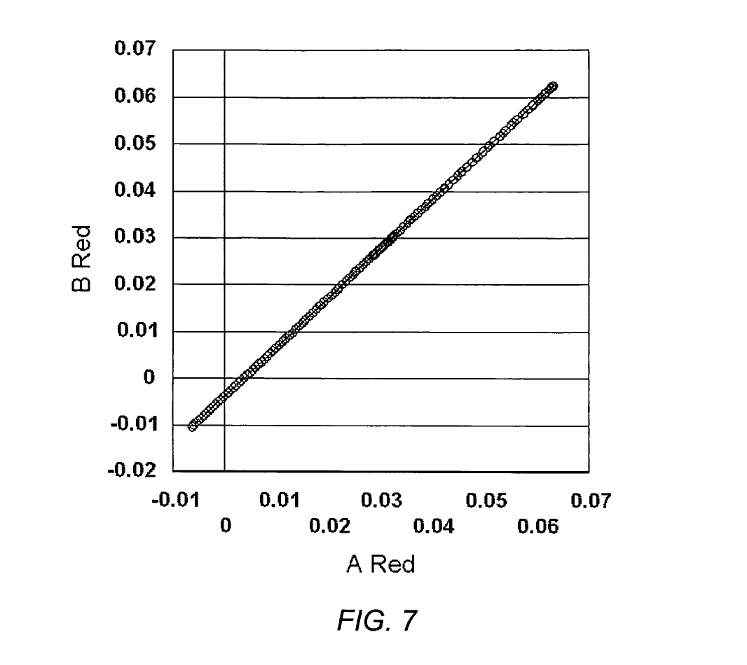

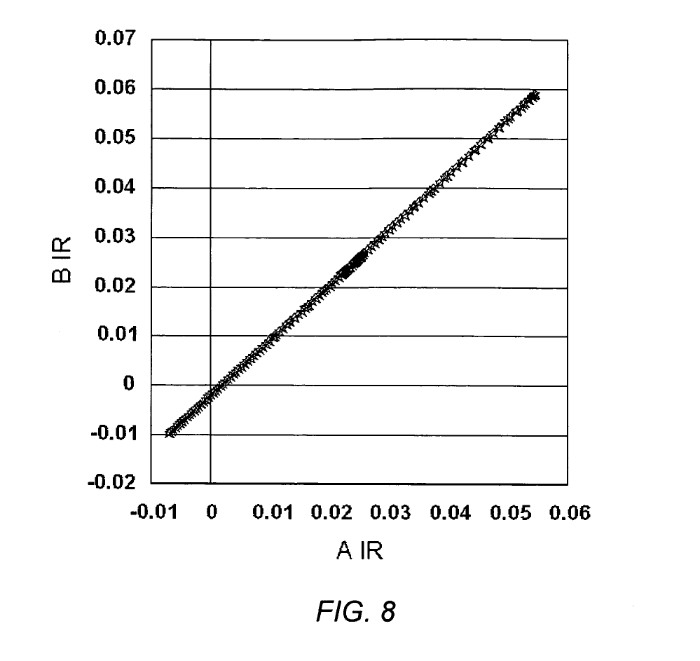

Data collected from independent sensors A and B, as shown in FIGS. 5 and 6, were corrected using the orthogonal regression routine described above with slope and offset derived for the actual data sets. The results of the orthogonal regression are shown in FIG. 7, which shows the corrected red signal data from sensor A graphed against the corrected red signal data from sensor B and FIG. 8, which shows the corrected infrared signal data from sensor A graphed against the corrected infrared signal data from sensor B. As can be seen in both referenced figures, the red data from each sensor and the infrared data from each sensor exhibit a more linear relationship after correction by orthogonal regression.

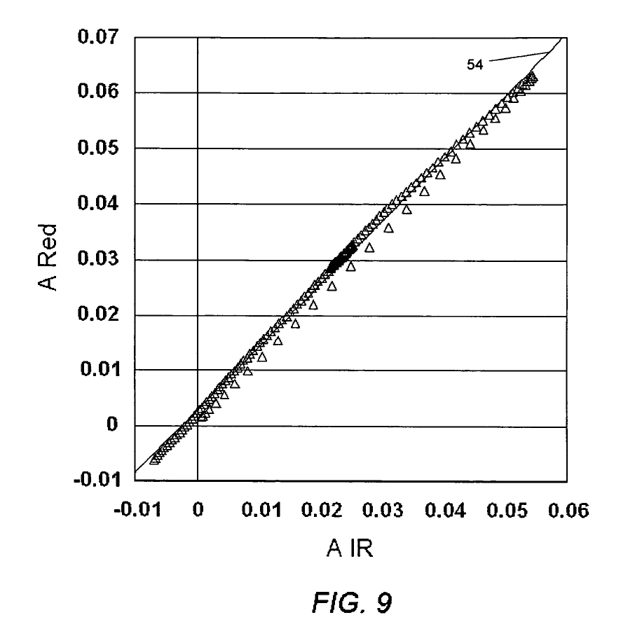

A similar process is preferably performed between the red and infrared signals from each sensor. FIG. 9 shows data corresponding to the red signal from sensor A during a single pulse graphed against the infrared signal during the same single pulse. As can be seen, the red and infrared signals from sensor A exhibit a relatively linear relationship, approximated by line 54. This data has an R-square equal to 0.996 with 176 data points and line 54 corresponds to the equation y=0.00274+1.14x.

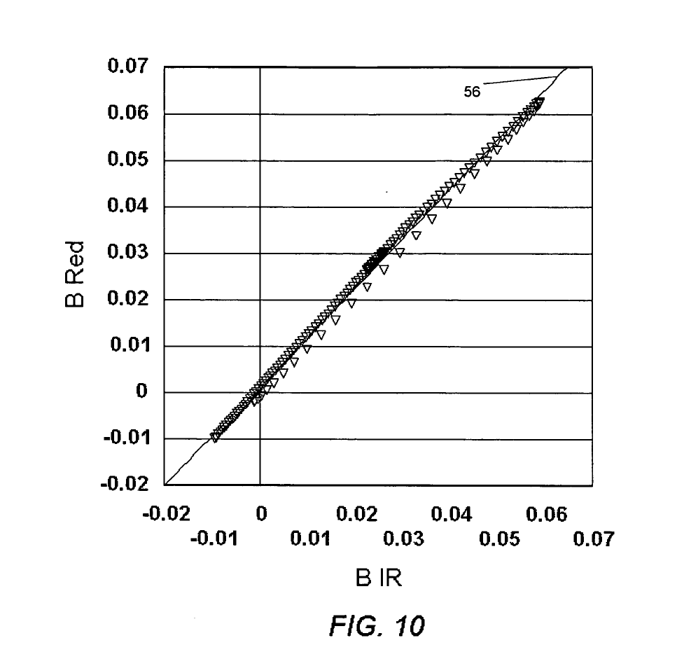

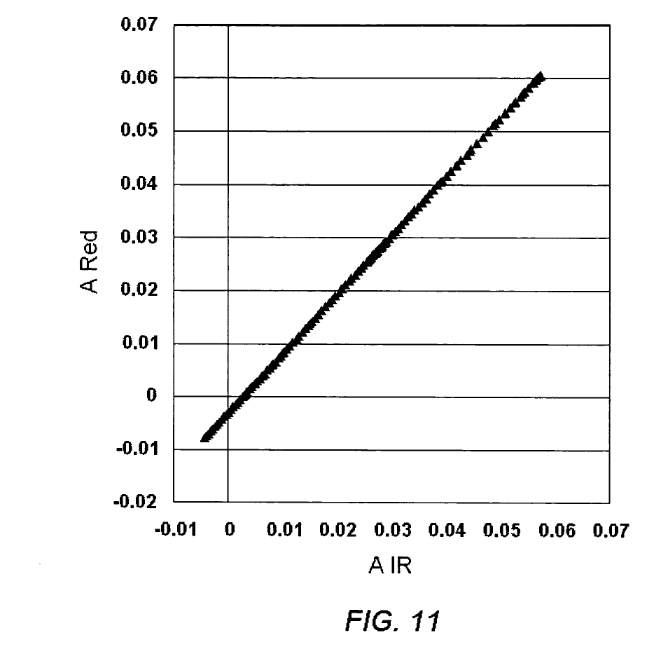

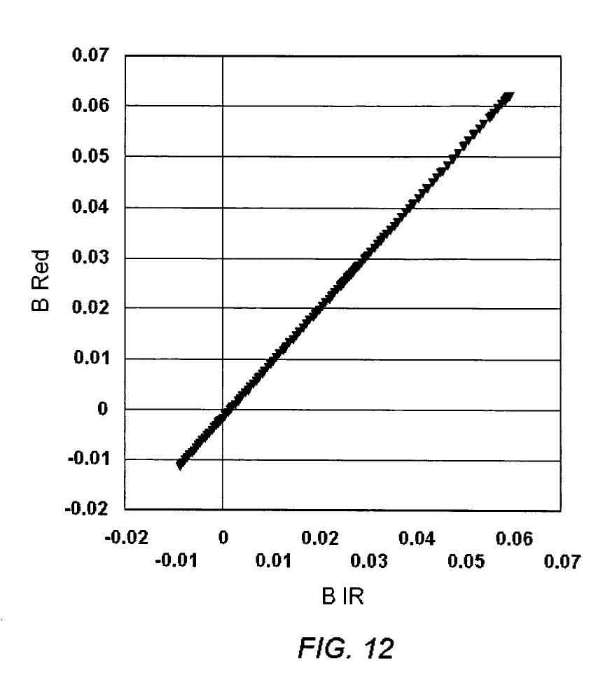

Similarly, FIG. 10 shows data corresponding to the red signal from sensor B during a single pulse graphed against the infrared signal. As with the data from sensor A, the red and infrared signals from sensor B exhibit a relatively linear relationship, approximated by line 56. This data has an R-square equal to 0.996 with 175 data points and line 56 corresponds to the equation y=0.00113+1.06x.

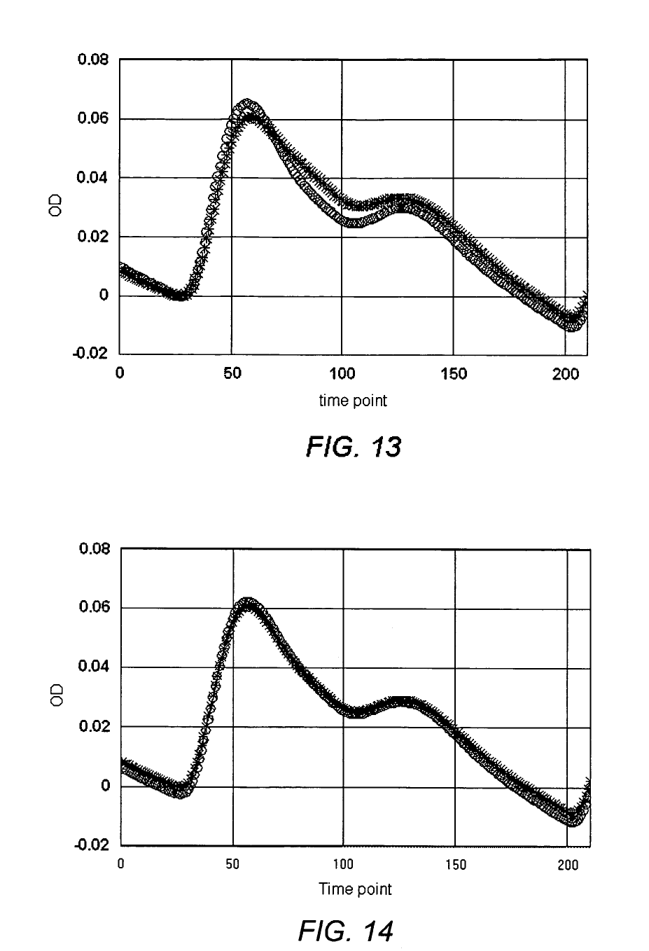

An orthogonal regression as described above is performed between the red signal and the infrared signal of each sensor. Thus, FIG. 13 shows the orthogonal regression of the red data set with the infrared data set of sensor A and FIG. 14 shows the orthogonal regression of the red data set with the infrared data set of sensor B. As can be seen, the corrected signals have slope and offset derived for the actual data sets and fall onto a single line as described by the same linear equation.

The orthogonal regression of the red and infrared signals from the two sensors yields four corrected data sets: A_{RedCorr}, A_{IRCorr}, B_{RedCorr} and B_{IRCorr}. The difference of these to their respective original data set, such as A_{RedCorr} minus the original red signal from sensor A, represents the amount removed in the process. The percent noise relative to signal removed is different at every time point for every data set. Although an indeterminable portion of desirable signal is also removed, the maximum amount typically removed by this process is 5 to 15% of total original signal.

The improvements represented by this invention can be demonstrated by the comparison of the original signal to the corrected signal. FIG. 13 shows data corresponding to the red channel data of both sensors during a single pulse before data correction by orthogonal regression. The open circles data points correspond to the signal from sensor A and the asterisk data points correspond to the signal from sensor B. As can be seen, there is considerable deviation between the values from each sensor, particularly in the range between approximately time point 50 and time point 100. In contrast, FIG. 14 shows data corresponding to the red channel signal from both sensors after data correction by the orthogonal regression described above. It is readily apparent that the data pairs of the corrected signals track each other significantly more closely after the orthogonal regression process. For example, there is tight correlation between the data pairs in the range of time point 50 to time point 100 where the uncorrected data showed significant deviation.

According to the invention, the corrected amplitudes A_{IRCorr} and A_{RedCorr} can be used to calculate a ratio of logarithms separately for each sensor. While other transforms can be used, the ratio of logarithms is the principal measurement parameter related to reference saturation percent for calibrating pulse oximeters and is thus preferred. The ratio R is then calculated as the absolute logarithm of the zeroed red amplitude over the absolute logarithm of the zeroed infrared amplitude:
R=|(log(A_{RedCorr}−A_{RedCorrMin}))|/|(log A_{IRCorr}−A_{IRCorrMin}))|(10)
The resulting ratio R is then related to the reference oxygen saturation conventionally as determined by CO-oximetry data.

Thus, in one embodiment of the invention, the physiological characteristic being measured is arterial oxygen saturation. In the noted embodiment, the first wavelength is preferably in the red light range and the second wavelength is preferably in the infrared light range. Thus, an orthogonal regression is first performed between the red signals of the first and second sensors and the infrared signals of the first and second sensors. A subsequent orthogonal regression is performed between the red and infrared signals of the first sensor and between the red and infrared signals of the second sensor. The orthogonal regressions generate corrected red and infrared signals from each sensor, representing a weighted average of the signals. A ratio of logarithms can be calculated from the corrected signals. This ratio is then related to reference oxygen saturation in a conventional manner. The orthogonal regression yields corrected signals having enriched oximetry signal content and an improved signal to noise ratio.

Additionally, the output of any appropriate plethysmographic data pre-treatment method can be advantageously used as input to the algorithmic data pretreatment method described in here in that the power of different and independent noise reduction methods will yield the best possible oximetry data.

In a further aspect of the invention, an acceptance criterion may be set to optimize the use of signal data. For example, the acceptance criterion can be based on the signal to noise of each independent sensor to determine whether the final ratio of logarithm results are averaged. Alternatively, the acceptance criterion can be based on the uncorrected ratio of logarithms from the individual sensors at a pre-determined pulse amplitude individual sensors. In one embodiment, the pre-determined pulse amplitude is the maximal value. In a further embodiment, the results of the two sensors are combined by averaging when the acceptance criterion is met. In one preferred embodiment, the ratios are combined when a delta of R is less than approximately 0.05. It is also preferable to apply weights, multiplying factors, before averaging individual ratios of logarithms relative to the signal to noise of the individual data sets.

As one having skill in the art will appreciate, choosing a curve that best fits a given set of data points is only one aspect of a regression analysis technique. Suitable techniques also include fitting a model with deterministic components that function as predictors as well as stochastic components to compensate for error.

Although orthogonal regression is preferred, in other embodiments of the invention, other mathematical forms of regression may be used to correct the signals from the independent sensors. For example, suitable techniques that can be used in the practice of the invention include, without limitation, linear regression, logistic regression, Poisson regression, supervised learning, and unit-weighted regression. Another suitable technique is disclosed in Taberner, D. A. and Dufty, J. M. An Easier Alternative To Orthogonal Regression For Calculation Of International Sensitivity Indexes, J. Clin. Pathol., Vol. 48, pp. 901-903 (October, 1995), Houboyan, L. L. and Goguel, A. F., Procedure Of Reference Calibrated Plasmas For Prothrombin Time Standardisation, Thromb. Haemost., Vol. 69, p. 663 (1993), and which are incorporated herein by reference.

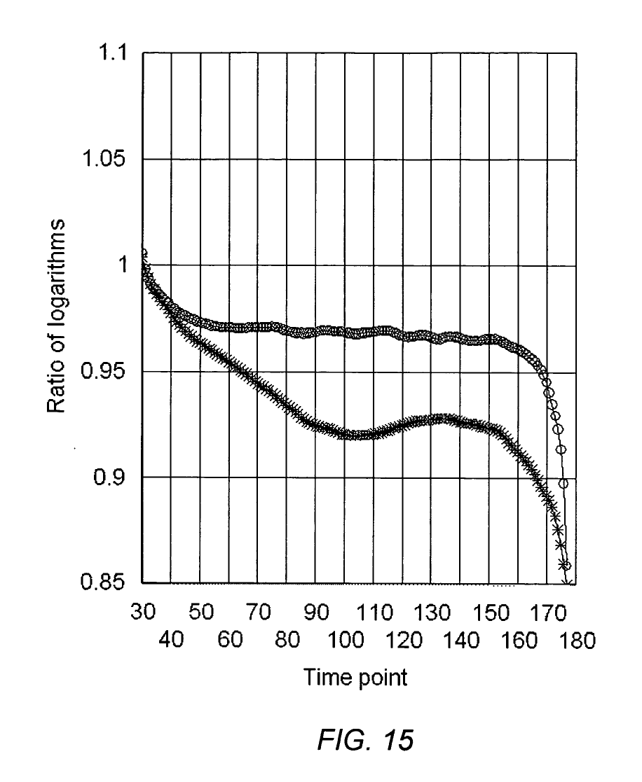

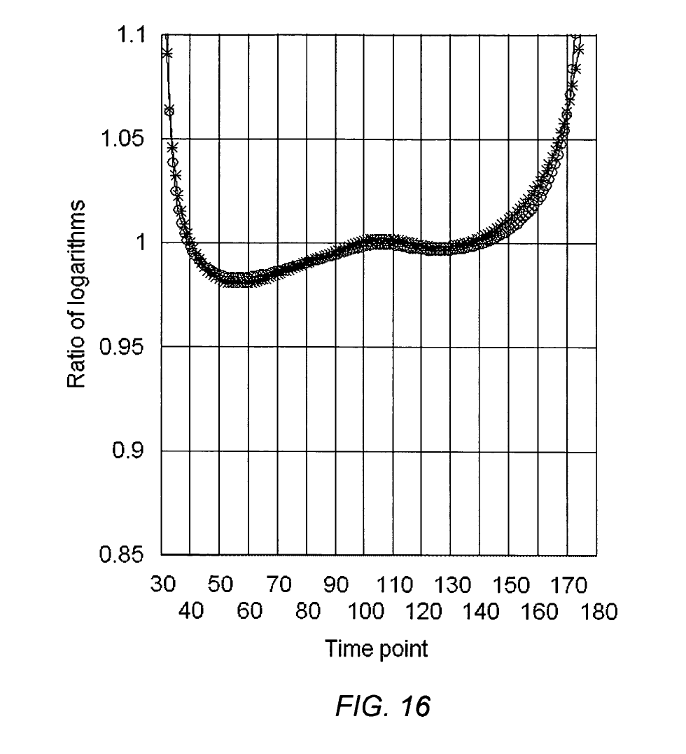

The described method also results in substantially reduced variability of the ratio of logarithms for each sensor during the same pulse. The difference between the two independent measurements from sensors A and B are markedly reduced even over the entire course of a single pulse wave. For example, FIG. 15 shows the ratio of logarithms calculated using data acquired from two independent sensors before processing by the sequential orthogonal regression process of the invention. The open circle data points correspond to one sensor while the asterisk data points correspond to the other sensor. As can be seen, there is significant deviation between the values calculated for each sensor over the entire curve. When the same data is subjected to correction by the orthogonal regression process of the invention, the ratio of logarithms calculated from each sensor is much closer. FIG. 16 shows the ratio of logarithms of same data from the two sensors after processing by the described sequential orthogonal regression. In contrast to FIG. 15, the data from each sensor yields nearly the same ratio throughout the curve.

One major advantage that the described data pre-processing method offers, is the unequivocal use of correction for different pulse amplitudes between different pulses and different patients as described in co-pending U.S. patent application Ser. No. 11/270,240, filed Nov. 8, 2005, which is hereby incorporated by reference in its entirety. Pulse amplitude may be calculated simply as the difference between the AC value at every time point minus the AC value at the ‘trough’ before the pulse wave relative to the DC value.

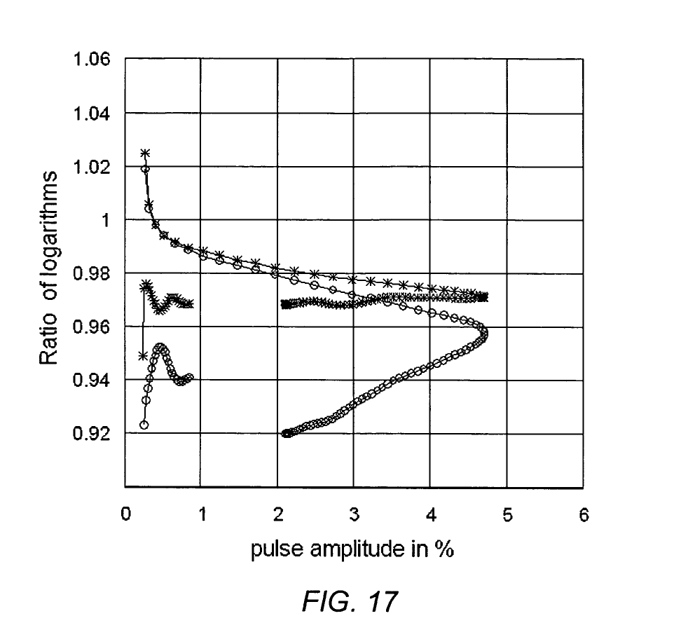

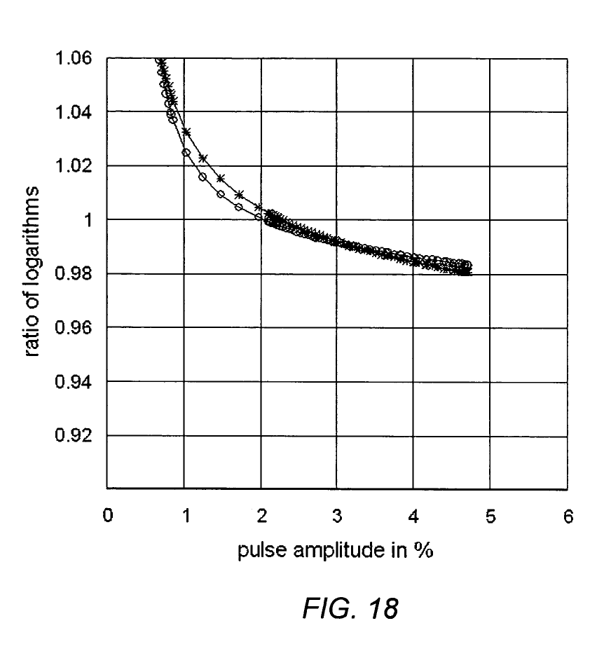

Referring now to FIG. 17 compares pulse amplitude to the ratio of logarithms calculated using data acquired from two independent sensors before processing by the sequential orthogonal regression process of the invention. The open circle data points correspond to data from one sensor while the asterisk data points correspond to the other sensor. As can be seen in FIG. 17, there is substantial scatter in the ratio of logarithms versus pulse amplitude in the uncorrected data. FIG. 18 shows the same comparison after the data is subjected to the sequential orthogonal regression correction process of the invention. As can be readily seen in FIG. 18, the deviation between the calculations derived from the two sensors is substantially minimized. Accordingly, pulse amplitude indexing to a known value as described in the above-identified patent application is significantly enhanced.

In one embodiment of the invention, the derived, corrected ratio of logarithms of the average value of both sensors may be expressed at 3% to eliminate pulse amplitude based error within and between patients.

Preferably, the original data is weighted prior to performing the orthogonal regression processes of the invention. More preferably, the data is weighted proportionally with pulse amplitude. In such embodiments, data fitting by standard least squares analysis provides a higher weighting for the higher pulse amplitudes because of their distance from the bulk of the data.

In another embodiment of the invention, the data is processed prior to regression by calculating relative weights. In this process, the raw regression weights are calculated by standard multiple regression analysis. If quadratic functions are used, standardized regression weights for these functions are obtained by combination. Finally, the combined weights are adjusted to provide relative weights. Further details regarding the calculation or relative weights are found in Hammond, K. R., Stewart, T. R., Brehmer, B., and Steinmann, D. Social Judgment Theory, Human Judgment and Decision Processes Formal and Mathematical Approaches, Kaplan, M. F. and Schwartz, S. (Eds.), p. 282 (New York: Academic Press, 1975), which is incorporated herein by reference.

In another embodiment of the invention, the data can be weighted by using a locally weighted linear regression to smooth the data. Generally, regression weights are calculated for the data within a given span, for example using a tricube function. A weighted linear least squares regression is then performed, using a first or second degree polynomial. Next, a smoothed value is obtained from the weighted regression at a given data point. If desired, a data sample having outliers can be subjected to the additional calculation of robust weights to minimize the influence of the outliers.

Alternatively, a modified form of a Pseudo Maximum Likelihood technique can be used in which the regression weights for point estimation of the model parameters provide a simple correction to the linearization variance estimators. Further details regarding this process are disclosed in Silva, N., Utilizing Auxiliary Information in Sample Survey Estimation and Analysis, Ph. D. Dissertation, Chap. 6, (University of Southampton, UK, 1996).

In further embodiments of the invention, the data can be weighted by analyzing regression characteristics using statistical diagnostic techniques including, without limitation, collinearity tolerance, Cook's Distance, DfFit and DfBeta. One having skill in the art will appreciate that other, known mathematical treatments for fitting data sets to provide preferential emphasis of data subsets that can readily be selected to achieve the desired results.

Furthermore, it is understood that subsets of a single pulse wave, such as only those data between a first AC minimum and the maximum or alternatively, only data down from the pulse amplitude maximum to the dichrotic notch, may be selected for the described error minimization by orthogonal regression.

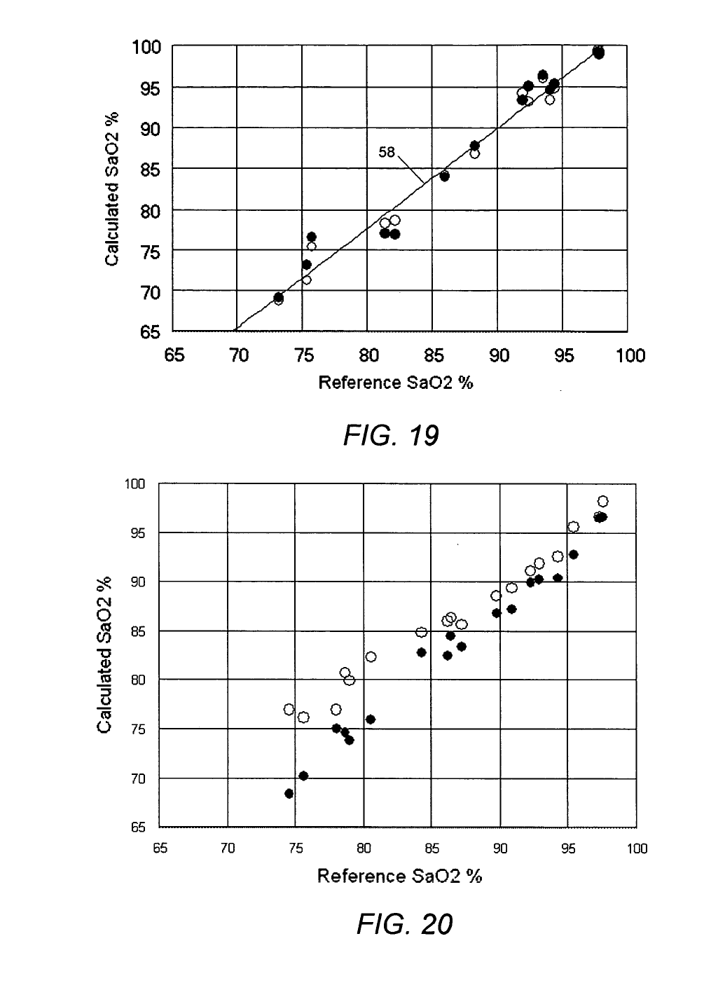

In one aspect of the invention, analysis of data from two independent sensors can reveal errors in measurement that could otherwise be undetected. For example, FIG. 19 shows high performance oximetry data collected simultaneously from two independent sensors. The signals have been processed according to the methods of the invention described above to result in a nearly complete removal of sensor to sensor differences. The closed circles indicate data from one sensor and the open circles from the other. Line 58 corresponds to reference CO-oximeter saturation. As can be seen in FIG. 19, the differences between data pairs from each sensor are, on average, closer to each other than to the reference CO-oximeter data. There is no direct physiological explanation for the similarity of two pulse oximetry data sets that are both different from the reference data. Thus, erroneous data entry or poor coordination of blood sampling presumably causes the remaining larger difference of most data points to the standard reference data. In such cases, the oximeter sensor data often can be trusted more than the reference data.

In another aspect of the invention, analysis of data from two independent sensors can reveal underlying physiological conditions. For example, FIG. 20 shows high performance oximetry data collected simultaneously from two independent sensors. The signals have been processed according to the methods of the invention described above to result in a nearly complete removal of sensor to sensor differences. The closed circles indicate data from one sensor and the open circles from the other. As can be seen in FIG. 20, the data pairs from the sensors are relatively close together at the higher saturations but get progressively further apart at lower saturations. Thus, the calculated saturations manifest a systematic drifting apart with decreasing saturation. This difference pattern is unlikely caused by erroneous data acquisition or transcription. Upon confirmation of correct instrument function and calibration, this comparison of sensor data presumably indicates a true physiological difference, conceivably caused by perfusion differences between the extremities. As can be appreciated by one of ordinary skill in the art, such a discrepancy between two simultaneous readings on the same patient can inform the attending physician of an underlying physiological difference between the two monitoring sites that needs to be taken into account for optimal saturation monitoring and patient care in general.

Accordingly, a comparison of oximetry data from at least two independent sensors preferably allows a determination of whether the data should be combined to improve precision or whether one stream of data can be rejected as being a less reliable indicator of the patient's oxygenation status. As an example of such a determination, the rapid change of calculated saturation in a first sensor while the second sensor continues to indicate unchanged saturation likely indicates the first sensor is suffering from interference.

In additional embodiments, the principles represented by the present invention can also be applied to a wide variety of other biological and physiological determinations. For example, U.S. Pat. Nos. 6,480,729, 6,537,225, 6,594,511, 6,719,705, 6,819,950, and 6,921,367 and U.S. patent application Ser. No. 10/912,721, filed 4 Aug. 2004, all of which are incorporated in their entirety by reference, each relate to the acquisition of signals for determining physiological characteristics and can be practiced with the methods and apparatus of the present invention.

For example, embodiments of the invention use signals from multiple sensors to improve the signal to noise ratio or to minimize artifacts. As such, sensors configured to determine any hemodynamic or blood based physiological parameter, including, but not limited to, cardiac output, blood pressure, ECG, blood pH, hemoglobin concentration and glucose concentration can be used in the practice of the invention.

Thus, a method of the present invention includes processing signals reflecting a physiological characteristic of a patient's blood, comprising the steps of i) coupling a first and second physiological sensor arrangement to independent tissue regions of the patient, ii) detecting a signal reflecting the physiological characteristic with each sensor arrangement, and computing the physiological characteristic of the patient's blood from corrected signals from each sensor arrangement determined by performing an error minimizing mathematical combination between the signal from the first sensor arrangement and the signal from the second sensor arrangement.

Without departing from the spirit and scope of this invention, one having ordinary skill in the art can make various changes and modifications to the invention to adapt it to various usages and conditions. As such, these changes and modifications are properly, equitably, and intended to be, within the full range of equivalence of the following claims.

What is claimed is:

1. A device for the monitoring of a physiological characteristic of a patient's blood, comprising:
a first sensor having a first radiation emitter that emits light at a first wavelength, a second radiation emitter that emits light at a second wavelength and a radiation detector configured to receive light at said first and second wavelengths after absorbance through the patient's blood and provide a first received intensity signal and a second received intensity signal corresponding to said first and second received wavelengths;
a second sensor having a first radiation emitter that emits light at a first wavelength, a second radiation emitter that emits light at a second wavelength and a radiation detector configured to receive light at said first and second wavelengths after absorbance through the patient's blood and provide a first received intensity signal and a second received intensity signal corresponding to said first and second received wavelengths; and
a controller for computing said physiological characteristic of said patient's blood from first corrected intensity signals from said first and second sensors and second corrected intensity signals from said first and second sensors;
wherein said corrected intensity signals are derived by performing an error minimizing mathematical combination between i) said first received intensity signal from said first sensor and said first received intensity signal from said second sensor, ii) said second received intensity signal from said first sensor and said second received intensity signal from said second sensor, iii) said first received intensity signal from said first sensor and said second received intensity signal from said first sensor, and iv) said first received intensity signal from said second sensor and said second received intensity signal from said second sensor.

2. The device of claim 1, wherein said error minimizing mathematical combination is orthogonal regression.

3. The device of claim 1, wherein said first and second corrected intensity signals are derived from a weighted average of said first and second received intensity signals.

4. The device of claim 1, wherein said physiological characteristic is arterial oxygen saturation.

5. The device of claim 4, wherein said first wavelength is in the range of approximately 650-670 nm.

6. The device of claim 5, wherein said second wavelength is in the range of 800-1000 nm.

7. The device of claim 1, wherein a ratio of logarithms of said first and second corrected intensity signals are related to reference oxygen saturation to determine said physiological characteristic.

8. The device of claim 1, wherein said first and second corrected intensity signals have an improved signal to noise ratio.

9. The device of claim 1, wherein a difference between said received intensity signals and said corrected intensity signals substantially corresponds to undesirable signal components.

10. The device of claim 1, wherein said controller indexes said received intensity signals to said patient's pulse amplitude.

11. The device of claim 7, wherein said controller indexes said received intensity signals to said patient's pulse amplitude and wherein said ratio of logarithms from said first sensor is averaged with said ratio of logarithms from said second sensor.

12. The device of claim 7, wherein said ratio of logarithms from said first sensor is averaged with said ratio of logarithms from said second sensor when a difference between said ratio of logarithms is below a desired acceptance criterion.

13. The device of claim 1, further comprising at least one additional sensor having a first radiation emitter that emits light at a first wavelength, a second radiation emitter that emits light at a second wavelength and a radiation detector configured to receive light at said first and second wavelengths after absorbance through the patient's blood and provide a first received intensity signal and a second received intensity signal corresponding to said first and second received wavelengths and wherein said controller computes said physiological characteristic of said patient's blood corrected intensity signals including corrected intensity signals from said additional sensor.

14. A method for processing signals reflecting a physiological characteristic of a patient's blood, comprising the steps of:
coupling a first and second oximeter sensor arrangement to independent tissue regions of said patient;
passing first and second lights through said patient's tissue region at each sensor arrangement, wherein said first light is substantially in a red light range and said second light is substantially in an infrared light range;
detecting said first and second lights absorbed by said tissue region and providing a first received intensity signal and a second received intensity signal corresponding to said absorbed first and second lights with each sensor arrangement; and
computing said physiological characteristic of said patient's blood from first corrected intensity signals from each sensor arrangement and second corrected intensity signals from each sensor arrangement determined by performing an error minimizing mathematical combination between i) said first received intensity signal from said first sensor arrangement and said first received intensity signal from said second sensor arrangement, ii) said second received intensity signal from said first sensor arrangement and said second received intensity signal from said second sensor arrangement, iii) said first received intensity signal from said first sensor arrangement and said second received intensity signal from said first sensor arrangement, and iv) said first received intensity signal from said second sensor arrangement and said second received intensity signal from said second sensor arrangement.

15. The method of claim 14, wherein said error minimizing mathematical combination comprises orthogonal regression.

16. The method of claim 14, wherein said corrected intensity signals are derived from a weighted average of said received intensity signals.

17. The method of claim 14, wherein said physiological characteristic is arterial oxygen saturation.

18. The method of claim 14, wherein a ratio of logarithms of said corrected intensity signals is related to reference oxygen saturation to determine said physiological characteristic.

19. The method of claim 14, further comprising the step of indexing said received intensity signals to said patient's pulse amplitude.

20. The method of claim 18, further comprising the steps of indexing said received intensity signals to said patient's pulse amplitude and averaging said ratio of logarithms from said first sensor is averaged with said ratio of logarithms from said second sensor.

21. The method of claim 18, further comprising the step of averaging said ratio of logarithms from said first sensor with said ratio of logarithms from said second sensor when a difference between said ratio of logarithms is below a desired acceptance criterion.

22. A method for processing signals reflecting a physiological characteristic of a patient's blood, comprising the steps of:
coupling a first and second physiological sensor arrangement to independent tissue regions of said patient;
detecting a signal reflecting said physiological characteristic with each sensor arrangement; and
computing said physiological characteristic of said patient's blood from corrected signals from each sensor arrangement determined by performing an error minimizing mathematical combination between said signal from said first sensor arrangement and said signal from said second sensor arrangement.
